(19) United States
(12) Patent Application Publication
(10) Pub. No.: US 2003/0189688 A1
(43) Pub. Date: Oct. 9, 2003

(54) Method of sizing an anterior chamber implant using optical coherence tomography

(76) Inventor: Mutlu Karakelle, Fort Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/115,716

(22) Filed: Apr. 3, 2002

(51) Int. Cl. A61B 3/10

(52) U.S. Cl. 351/205

(57) ABSTRACT

A method for measuring anterior chamber dimensions using optical coherence tomography. Such a method is more accurate and reliable in determine anterior chamber angle diameter and other anterior chamber dimensions.

### BACKGROUND OF THE INVENTION

This invention relates generally to the field of intraocular lenses (IOL) and, more particularly, to anterior chamber phakic IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

The optical power of the eye is determined by the optical power of the cornea and the crystalline lens. In the normal, healthy eye, sharp images are formed on the retina (emmetropia). In many eyes, images are either formed in front of the retina because the eye is abnormally long (axial myopia), or formed in back of the retina because the eye is abnormally short (axial hyperopia). The cornea also may be asymmetric or toric, resulting in an uncompensated cylindrical refractive error referred to as corneal astigmatism. In addition, due to age-related reduction in lens accommodation, the eye may become presbyopic resulting in the need for a bifocal or multifocal correction device.

In the past, axial myopia, axial hyperopia and corneal astigmatism generally have been corrected by spectacles or contact lenses, but there are several refractive surgical procedures that have been investigated and used since 1949. Barraquer investigated a procedure called keratomileusis that reshaped the cornea using a microkeratome and a cryolathe. This procedure was never widely accepted by surgeons. Another procedure that has gained widespread acceptance is radial and/or transverse incisional keratotomy (RK or AK, respectively). Recently, the use of photablative lasers to reshape the surface of the cornea (photorefractive keratectomy or PRK) or for mid-stromal photoablation (Laser-Assisted In Situ Keratomileusis or LASIK) have been approved by regulatory authorities in the U.S. and other countries. All of these refractive surgical procedures cause an irreversible modification to the shape of the cornea in order to effect refractive changes, and if the correct refraction is not achieved by the first procedure, a second procedure or enhancement must be performed. Additionally, the long-term stability of the correction is somewhat variable because of the variability of the biological wound healing response between patients.

Several companies are investigating implantable anterior chamber phakic IOLs, including Bausch & Lomb's NuVita and Model ZB5M lenses, and the Artisian iris claw lens by Ophtec BV. These and other anterior chamber phakic lenses are described in U.S. Pat. No. 5,071,432 (Baikoff), U.S. Pat. No. 5,192,319 (Worst), U.S. Pat. No. 5,300,117 (Baikoff, et al.), U.S. Pat. No. 5,928,282 (Nigam) and PCT Publication No. WO 98/56315, the entire contents of which being incorporated herein by reference. The clinic experience with commercially available anterior chamber phakic lenses has not been entirely satisfactory due to difficult implantation techniques and clinical complications such as endothelial cell loss, lens displacement and pupil ovaling. Lens displacement and pupil ovaling are primarily the result of improperly sized implants. In the past, to size the implant (i.e., identify the overall diameter of the implant), diameter of the anterior chamber angle was estimated by taking the measurement of the diameter of the sclera at the corneal junction (the so-called “white-to-white” measurement). To this measurement, some additional adjustment was made. Usually, the implant was sized at white-to-white plus 1 millimeter, or some fraction of a millimeter was added to the white-to-white measurement. See, for example, WIPO Publication No. WO 01/70098, the entire contents of which being incorporated herein by reference. This measurement technique, however, has proven to be unreliable and not consistently predictive of the proper implant size.

Therefore, a need continues to exist for an accurate and reliable method to measure the diameter of the anterior chamber angle and other anterior chamber measurements.

### BRIEF SUMMARY OF THE INVENTION

The present invention improves upon the prior art by providing a method for measuring anterior chamber dimensions using optical coherence tomography. Such a method is more accurate and reliable in determine anterior chamber angle diameter and other anterior chamber dimensions.

Accordingly, one objective of the present invention is to provide an accurate method of measuring anterior chamber dimensions.

Another objective of the present invention is to provide a reliable method of measuring anterior chamber dimensions.

Still another objective of the present invention is to provide a method of measuring anterior chamber dimensions using optical coherence tomography.

These and other advantages and objectives of the present invention will become apparent from the detailed description and claims that follow.

### BRIEF DESCRIPTION OF THE DRAWING

FIG. 1 is a perspective view of an ophthalmic implant that can be used with the method fo the present invention.

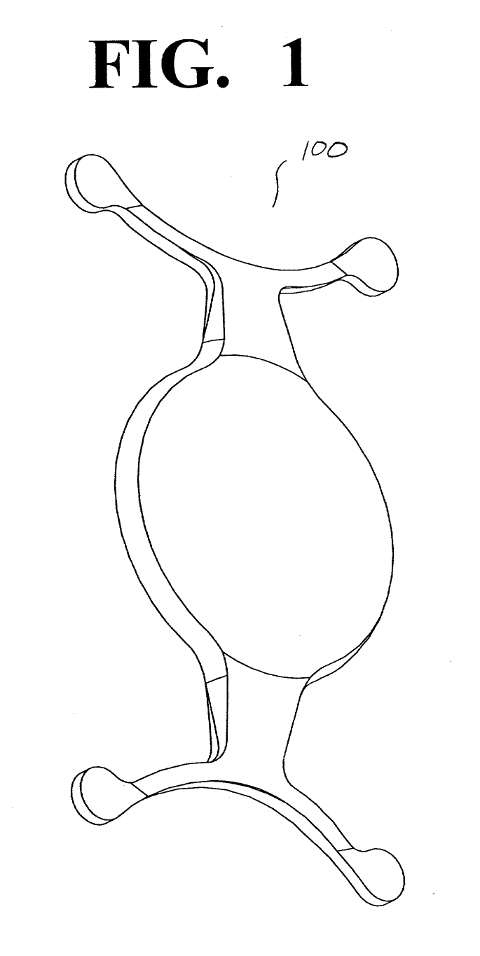

FIG. 2 is a representative image of an eye generated using optical coherence tomography.

### DETAILED DESCRIPTION OF THE INVENTION

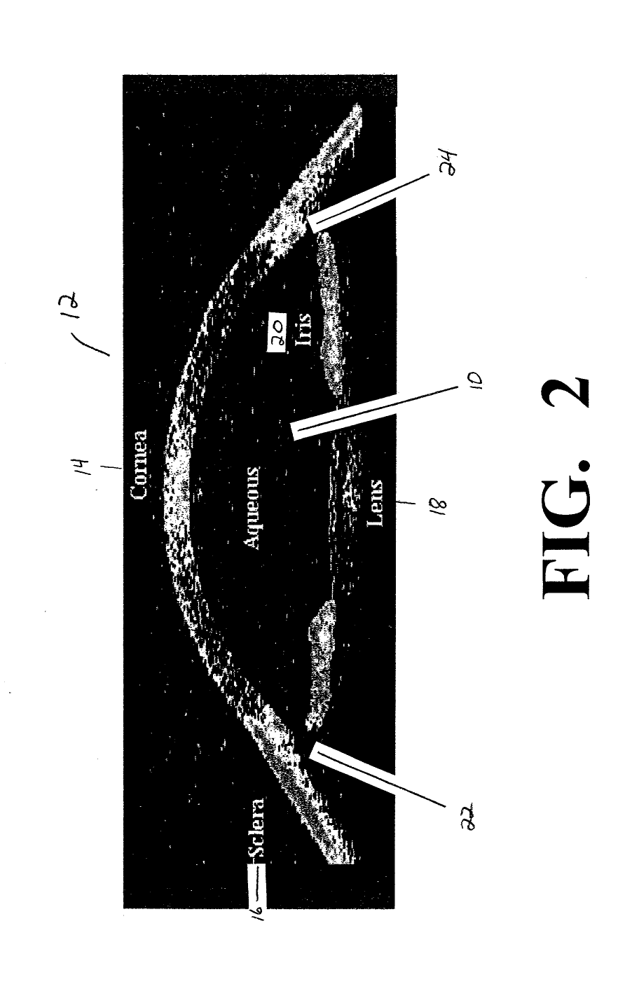

As seen in FIG. 2, optical coherence tomography (“OCT”) can be used to provide high quality images of anterior chamber 10 of eye 12. Structures such as cornea 14, sclera 16, lens 18, iris 20 and anterior chamber angles 22 and 24 can be identified readily. OCT systems are well-known to those skilled in the art and commercially available from Humphrey Instruments. A device using a longer wavelength, such as 1310 nanometers may also be used and may provide enhanced light penetration. In addition, this longer wavelength light may be safer to the retina. Other systems are also disclosed in U.S. Pat. No. 5,093,833 (Pang, et al.), U.S. Pat. No. 5,321,501, U.S. Pat. No. 5,459,570 (Swanson, et al.), U.S. Pat. No. 5,553,093 (Ramaswamy, et al.) and Pat. Nos. 5,553,093, 6,111,645, 6,282,001 (Tearney, et al.), the entire contents of which being incorporated herein by reference.

In use, the method of the present invention includes acquiring an OCT image of the anterior chamber of the eye similar to the image illustrated in FIG. 2. The operator, through appropriate software control, can highlight the location of anterior chamber angles 22 and 24 and a computer (not shown) can calculate the distance between angle location 22 and location 24, thereby helping to size accurately anterior chamber implant 100. The optimum overall diameter of implant 100 in relation to the diameter of the anterior chamber angle will vary according to the design and material used for implant 100, but may be easily determined once the diameter of the anterior chamber angle is accurately measured.

This description is given for purposes of illustration and explanation. It will be apparent to those skilled in the relevant art that changes and modifications may be made to the invention described above without departing from its scope or spirit.

I claim:

1. A method for sizing an anterior chamber implant, comprising the steps of:
a) measuring an anterior chamber of an eye using optical coherence tomography;
b) determining the overall diameter of the anterior chamber measured at an anterior chamber angle by using data provided by the optical coherence tomography measurement of the anterior chamber; and
c) calculating the optimal size of an anterior chamber implant using at least the overall diameter of the anterior chamber as determined by the optical coherence tomography measurement of the anterior chamber.

2. The method of claim 1 wherein the anterior chamber implant is a phakic intraocular lens.
